(19) United States
(12) Patent Application Publication
(10) Pub. No.: US 2014/0121191 A1
(43) Pub. Date: May 1, 2014

(54) Oral Energy Supplement and Related Methods

(71) Applicants: Robert Davidson, Woodland Hills, CA (US); Eric Allen, Camarillo, CA (US); Edward Maliski, Thousand Oaks, CA (US); Jose Bernado, Thousand Oaks, CA (US)

(72) Inventors: Robert Davidson, Woodland Hills, CA (US); Eric Allen, Camarillo, CA (US); Edward Maliski, Thousand Oaks, CA (US); Jose Bernado, Thousand Oaks, CA (US)

(21) Appl. No.: 13/850,292

(22) Filed: Mar. 25, 2013

Related U.S. Application Data
(60) Provisional application No. 61/647,867, filed on May 16, 2012.

(51) Int. Cl. A61K 31/555; A61K 31/045; A61K 45/06; A61K 31/44; A61K 31/4985; A61K 31/455; A61K 31/522

(52) U.S. Cl. CPC A61K 31/555; A61K 31/455; A61K 31/045; A61K 31/522; A61K 31/44; A61K 31/4985; A61K 45/06
USPC 514/185; 514/356; 514/263.34; 514/277; 514/249; 514/263.31

(57) ABSTRACT

The present invention relates to oral dosage formulations consisting of at least one active ingredient contained in a plurality of hydrophobic carriers dispersed in an aqueous medium comprising a hydrocolloid. The active ingredients include various energy supplements, such as caffeine and vitamins. The formulations may further include emulsifiers, permeation enhancers, and vasodilators. The invention further relates to methods of making the oral dosage formulations.

### CROSS-REFERENCE TO RELATED APPLICATION

This application claims the benefit of U.S. provisional application No. 61/647,867, filed May 16, 2012, titled “Oral Liposomal Energy Supplement Solution and Related Methods,” which is incorporated by reference herein in its entirety.

### FIELD OF THE INVENTION

The present invention relates to the field of dietary supplements, and, more particularly, to an oral energy supplement solution and related methods.

### BACKGROUND OF THE INVENTION

Caffeine and B vitamin complexes, when taken orally, are reported to improve cognitive performance and increase the feeling of alertness. Improvements in physical activity, performance, and muscular endurance are also reportedly improved by caffeine and B vitamin complexes, therefore, benefiting athletes and those individuals participating in athletic endeavors.

Caffeine is a natural central nervous system stimulant for mammals, and is known to promote alertness and reduce physical fatigue. Athletes have reported improvement in both endurance and sprinting occurs as a result of caffeine ingestion. After oral consumption, the physical effects of caffeine are typically felt within an hour.

B vitamins are essential to numerous functions within the human body. For example, niacin (vitamin B3) is associated with DNA repair, the production of steroid hormones, and is a precursor to NAD+/NADH and NADP+/NADPH; pyridoxine (vitamin B6) is required for neurotransmitter production (dopamine, serotonin, epinephrine, and norepinephrine); and folic acid (vitamin B9) is essential for DNA repair, synthesis, and methylation and is also implicated in aiding cellular division and growth. In terms of symptoms, a deficit in B vitamins is related to fatigue, irritability, and poor concentration.

Energy drinks on the market often contain both caffeine and B vitamins, but for these compounds to be efficacious the body must process them. Until caffeine and B vitamins pass from the digestive tract into the bloodstream, these compounds are of little value to the body. Therefore, it is desirable for such energy supplements to be absorbed by the body as rapidly and efficiently as possible.

### SUMMARY OF THE INVENTION

In view of the foregoing background, it is therefore an object of the present invention to provide an energy supplement formulation containing caffeine and B vitamins that is quickly and efficiently absorbed by the body. This and other objects, features, and advantages in accordance with the present invention are provided by an encapsulated and/or colloidally suspended energy supplement formulation for oral administration.

Another aspect is directed to methods for producing a colloidal and/or liposomal energy supplement formulations.

Another aspect is directed to a method for administering a liposome encapsulated or colloidally suspended energy supplement formulation to an individual in need thereof.

These and other objects, aspects, and advantages of the present invention will be better appreciated in view of the drawings and the following detailed description of the embodiments.

### BRIEF DESCRIPTION OF THE DRAWINGS

FIG. 1 is a diagram illustrating a phospholipid molecule;

FIG. 2 is a diagram illustrating active ingredients within a micelle; and

FIG. 3 is a diagram illustrating active ingredients within a liposome.

### DESCRIPTION OF EMBODIMENTS OF THE INVENTION

In the Summary of the Invention above and in the Detailed Description of the Invention and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

In this section, the present invention will be described more fully with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will convey the scope of the invention to those skilled in the art.

The inventors have developed formulations that can be used to orally deliver liposome-encapsulated, micelle-encapsulated, and/or colloidally suspended active ingredients. A formulation in accordance with an embodiment of the invention comprises one or more active ingredients, preferably caffeine and a B vitamin complex, encapsulated in micelle or liposome carriers or suspended in a hydrocolloid solution. A hydrocolloid is a chemical substance in which colloidal particles are spread throughout water.

As used herein, the term “active ingredient” means an ingredient in the formulation that produces a physiological effect in the user. A preferred concentration range of active ingredients is about 1% (w/w) to about 15% (w/w) of the formulation. However, the concentration range may be expanded or contracted, depending on the particular active ingredient or combination of active ingredients.

Because the formulations can be administered to a subject in need thereof, one having ordinary skill in the art will recognize that the formulations can further contain substances used for the preparation of a final dosage form as is readily understood in the pharmaceutical and nutraceutical arts. Examples of these substances include one or more excipients, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, essential oils, emollients, dissolution enhancers, dispersing agents, or combinations thereof.

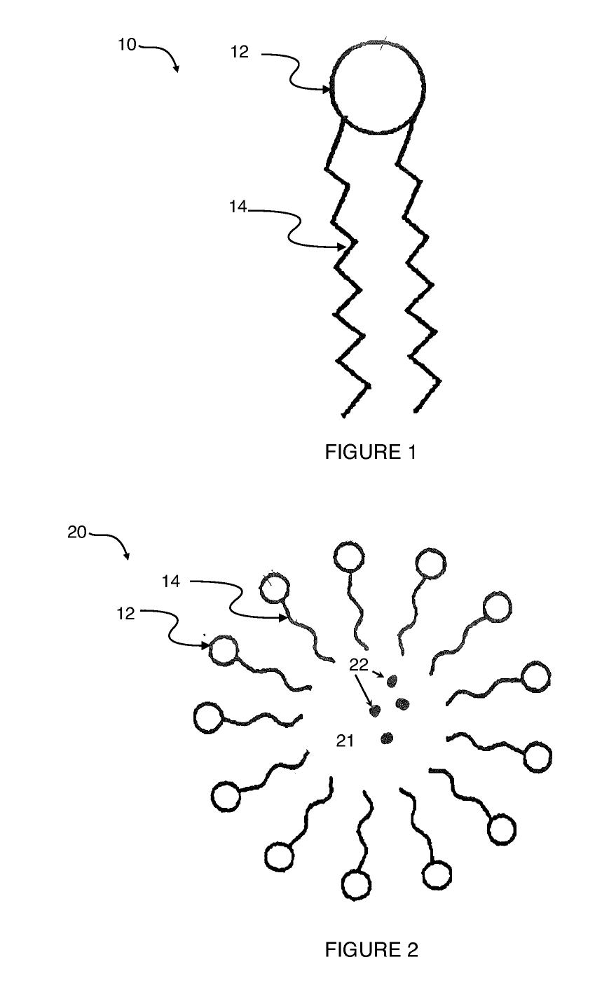

FIG. 1 illustrates a phospholipid 10, which is a type of amphiphilic lipid capable of forming lipid bilayers. Typically, phospholipids 10 comprise a phosphate head 12 and a hydrophobic tail 14. The phosphate head 12 is largely hydrophilic.

FIG. 2 illustrates a micelle 20 formed from amphiphilic molecules, such as phospholipids 10 (FIG. 1). When dispersed in an aqueous solution, the hydrophilic head regions 12 form a pocket 21 around the hydrophobic tail region 14. One or more active ingredients 22 may be encapsulated in the micelle 10.

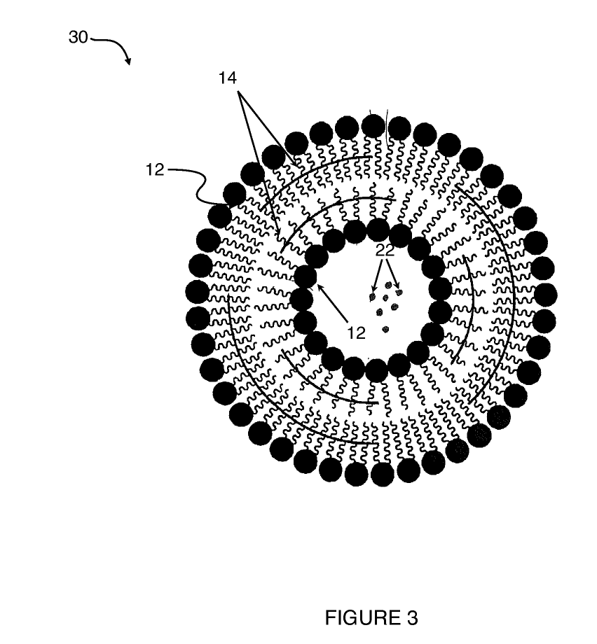

FIG. 3 illustrates a liposome 30, which is an artificially prepared vesicle similar to a micelle 20, but composed instead of a lipid bilayer. Many substances that form micelles 20 and liposomes 30 are known. Examples of these substances include, but are not limited to, both natural and synthetic phosphatidyl-based substances including lecithins (phosphatidylcholines), hydroxylated lecithin, PEG phospholipid, hydrogenated soy phosphatidylcholine, phosphatidic acid, phosphatidylglycerol, phosphatidylethanolamine, phosphatidyl serine; sulfolipids such as sulfoquinovosyl distearoylglycerol; sulfates such as sodium lauryl sulfate; sulfonates such as dioctyl sodium sulfosuccinate; and carboxylates such as sodium deoxycholate, sodium stearate, and sodium oleate.

In at least one embodiment, phosphatidyl serine is first dispersed throughout an aqueous solution to begin to form carrier micelles 20 and liposomes 30 within the solution. The micelles 20 or liposomes 30 used in the composition of the invention are preferably electrically charged. This is accomplished by choosing micelle- or liposome-forming substances that can comprise ionic constituents. Phospholipids 10, for example, can be treated chemically in order to provide anionic phosphate-based functional groups in the hydrophilic head 12 region. Phosphatidyl serine in particular can be made into a mono- or di-anion by treating it with one or two equivalents of a base. Positively charged counter ions are solvated and distributed in the aqueous solution external to the liposome 30.

In a preferred embodiment, sodium hydroxide (NaOH) is introduced to the phosphatidyl serine, and the hydroxide deprotonates the carboxyl group of the phosphatidyl serine, giving the hydrophilic part of the carrier liposomes a negative charge. The phosphatidyl serine and NaOH are mixed at high speed in water to fully disperse carrier liposomes 30 throughout the water so that the hydroxide evenly interacts with all of the carrier micelles or liposomes 30. The amount of sodium hydroxide is about a 1:1 molar ratio with phosphotidyl serine. It is important that each phosphatidyl serine molecule be exposed to a hydroxide molecule in order for all, or the majority, of the carrier liposomes 30 to become negatively charged, so that overall, the surface of the carrier liposomes 30 will have a net negative charge. It is also important to first create the negative charge on the surface of the liposomes 30 or micelles 20 before introducing the active ingredients 22 (FIGS. 2 and 3) because the active 22 may interfere with the hydroxides' de-protonation of the carboxyl group of the phosphatidyl serine.

The active ingredients 22 are, among other substances, compounds that stimulate energy levels, alertness, and cognitive performance. The active ingredients 22 of the formulation can be selected, for example, from the group comprising methylxanthines, caffeine, vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid), vitamin B12 (cyanocobalamin), guarana, yerba mate, acai, taurine, ginseng, maltodextrin, inositol, carnitine, creatine, glucuronolactone, ginkgo biloba, and combinations thereof. In some embodiments, the formulation comprises about 1% (w/w) to about 5% (w/w) anhydrous caffeine; about 0.05% (w/w) to about 0.1% (w/w) niacin; about 0.5% (w/w) to about 5% (w/w) vitamin B6; about 0.005% (w/w) to about (w/w) 0.05% folic acid; and about 0.005% (w/w) to about 0.05% (w/w) vitamin B12. In a preferred embodiment, the formulation comprises about 3.5% (w/w) anhydrous caffeine; about 0.1% (w/w) niacin; about 2.0% (w/w) vitamin B6; about 0.02% (w/w) folic acid; and about 0.025% (w/w) vitamin B12. In total, the combination of active ingredients may make up from about 1% (w/w) to about 15% (w/w) of the formulation.

These active ingredients 22 are dispersed in hydrophobic carriers, forming either micelles, liposomes, or oil droplets, in a colloidal suspension comprising a hydrocolloid for delivery through the buccal mucosa and gastrointestinal tract into the blood stream.

The hydrocolloid is a hydrogel that forms the aqueous medium around the hydrophobic carriers. Non-limiting examples of hydrocolloids include cellulose derivatives such as hydroxyethylcellulose, methylcellulose, and hydroxypropyl-methylcellulose; agarose; hyaluronan; pectin; locust bean gum; carrageenans; gellan gums; polyvinyl alcohol; pullulan; and other polymers either synthetically or naturally derived. A particularly preferred gelling agent is xanthan gum. A preferred concentration range of gelling agent is about 0.50% (w/w) to about 6.0% (w/w) of the formulation.

In embodiments in which the oral formulation comprises a colloidal suspension, the hydrophobic carriers are lipophilic particles or droplets suspended in the aqueous medium. Such hydrophobic carriers can be formed from almond oil, argan oil, avocado oil, canola oil, cashew oil, castor oil, coconut oil, cod liver oil, colza oil, corn oil, cottonseed oil, fish oil, hazelnut oil, hemp oil, linseed oil (flaxseed oil), macadamia oil, marula oil, mongongo nut oil, mustard oil, olive oil, palm oil (palm kernel oil), peanut oil, pecan oil, perilla oil, pine nut oil, pistachio oil, poppy seed oil, pumpkin seed oil, grapeseed oil, rice bran oil, safflower oil, sesame oil, soybean oil, sunflower oil, tea seed oil, walnut oil, watermelon seed oil, and combinations thereof. Grapeseed oil is particularly preferred due to its relatively low viscosity.

Preferably, the diameter of the suspended hydrophobic carriers is small enough to pass through pores of the walls of the mouth and tongue.

The hydrophobic carriers may also include lecithin, phosphatidylserine and combinations thereof. The hydrophobic carriers generally comprise from about 0.5% (w/w) to about 3% (w/w) of the formulation.

Optionally, the formulations may include one or more emulsifiers to prevent the carriers from agglomerating and settling into a continuous oil phase. The use of an emulsifier is more important in the colloidal suspensions. Suitable emulsifiers include, but are not limited to, lecithin, hydroxylated lecithin, glycerin, sodium stearoyl lactylate, cetearyl alcohol, polysorbates, polyoxyethylene ethers, polyethylene glycol, anisolic compounds, any conventional emulsifier, and combinations thereof. Glycerin is a particularly preferred emulsifier due to its relatively low viscosity. A preferred concentration range of emulsifier is about 1% (w/w) to about 3% (w/w) of the composition.

Optionally, the formulations may include one or more preservatives for preventing the formulation from spoiling. Suitable preservatives include, but are not limited to, antimicrobial preservatives and antioxidants. Examples include sorbic acid and its salts, benzoic acid and its salts, calcium propionate, sodium nitrite, natamycin, sodium nitrate, sulfites, sulfur dioxide, sodium bisulfite, potassium hydrogen sulfite, disodium EDTA, butylated hydroxyanisole, butylated hydroxytoluene, tert-butylhydroquinone, propyl gallate, ethanol, and methylchloroisothiazolinone. Sodium benzoate and potassium sorbate are particularly preferred preservatives. A preferred concentration range of preservative is about 0.01% (w/w) to about 0.05% (w/w) of the composition.

The formulations may also include one or more vasodilators to assist the active ingredient(s) in permeating the buccal cell layers. Examples of vasodilators include, but are not limited to, menthyl acetate, DDAIP, fatty acid esters, fatty alcohol ethers, ethanol, dimethylsulfoxide, polyethylene glycol monolaurate, sesquiterpenes, terpenoids, sesquiterpenoids, and terpenes such as menthol, peppermint oil, nerolidol, nerol, linalool, citronellol, and geraniol; and combinations thereof. A particularly preferred vasodilator is menthol, as menthol also provides a pleasing taste for oral consumption. A preferred concentration range of vasodilator is about 0.2% (w/w) to about 7% (w/w) of the formulation.

Optionally, the formulations may include one or more vasostimulant to aid in ingredient delivery. Suitable vasostimulants include any conventional vasostimulant such as nicotinic acid, caffeine, and combinations thereof. A preferred concentration range of vasostimulant is about 0.2% (w/w) to about 7% (w/w) of the formulation.

It is important to blend the active ingredients 22 with the hydrophobic carriers of the hydrocolloid solution and/or encapsulating phospholipids 10, micelles 20, and liposomes 30 before adding ingredients for palatability. If the active ingredients 22 are to be encapsulated via micelles or liposomes, they must always be blended into the solution before the other ingredients to ensure they are the only molecules to be encapsulated within the liposome 30. A high speed blending operation will open the carrier micelles 20 and liposomes 30 to allow them 20, 30 to reform around, or encapsulate, molecules of the active ingredients 22. The solution is also allowed to slightly cool at this point to allow the matrix to settle and the liposomes 30 to fully re-form and encapsulate the active ingredients 22.

After the micelles 20 and liposomes 30 are formed around the active ingredients 22, the solution will no longer be blended at high speed. A high-speed blend will cause the carrier micelles 20 and liposomes 30 to re-open, which will allow the active ingredients 22 to be released from encapsulation.

After the active ingredients 22 are added to the solution, blended, and allowed to rest, glycerin is added to the solution. The glycerin helps emulsify the immiscible liquids to evenly distribute solution components throughout the solution. The remaining ingredients of the formulation are then added in succession. These ingredients will not be encapsulated in the carrier micelle 20 or liposome 30, or dispersed in the colloidal suspension if a colloidal is the desired form.

It is not desirable to open the carrier micelles 20 and liposomes 30 after the active ingredients 22 are encapsulated. The unencapsulated ingredients in the formulation are the first components exposed to the taste receptors and the buccal mucosa in the mouth. Allowing sodium, flavors, and sweeteners to enter into the liposomes would negate the purpose of such unencapsulated ingredients. For example, ingredients such as sweeteners and flavors that are incorporated specifically for palatability must be presented to the buccal mucosa, and should not be shielded from the mucosa by a layer of phospholipids 10. Sodium chloride, for example, also aids in the shuttling of liposomes 30 through the buccal mucosa by active transport.

Sugar substitutes, artificial and natural, are contemplated as sweeteners for the formulation. In particular, without limitation, stevia, aspartame, sucralose, neotame, acesulfame potassium, and saccharin may be used as sweetening agents. Natural sugar substitutes such as sorbitol and xylitol are also, without limitation, contemplated. In a preferred embodiment, sucralose, acesulfame potassium, xylitol and sodium chloride are used in combination to provide sweetness and palatability to the formulation.

The flavors contemplated for the formulation are, without limitation, almond, amaretto, amaretto nutty, anise, apple, apricot, banana creme, bavarian creme, bergamot, black walnut, blackberry, blueberry, brandy, bubble gum, butter, butter rum, butterscotch, cappuccino, caramel, champagne, cheesecake, cherry, cherry washington, chocolate, chocolate hazelnut, cinnamon, cinnamon roll, citrus blossom, clove, coconut, coffee, coffee keoke, coffee kona, cola, cotton candy, cranberry, cranraspberry, créme de menthe, eggnog, english toffee, ginger, grape, grapefruit pink, guava, hazelnut creamy, honey, honeydew, horchata, horehound, hot chili, irish cream, key lime, lavender, lemon, lemonade, licorice, lime, mango, maple, maple Canadian, marshmallow, melon, menthol eucalyptus, mint chocolate chip, mixed berry, mountain berry, nutmeg, orange brandy, orange cream, orange, peach, peanut butter, pear, pecan, peppermint, pina colada, pineapple, pistachio, plum, pomegranate, praline, pralines and cream, pumpkin, raspberry, red licorice, root beer, royal raspberry, salt water taffy, sassafras, spearmint, strawberry banana, strawberry, strawberry kiwi, tangerine, teaberry, tropical punch, tutti-frutti, vanilla butternut, vanilla, watermelon, wintergreen, any of which may be in an oil or solid form. In a preferred embodiment, the flavors in the formulation are honeydew, mixed berry, and mountain berry.

Sodium benzoate and potassium sorbate are preservatives that are added to the formulation for the purpose of keeping the solution fresh and to prevent bacteria from growing. Preservatives contemplated for the formulation include, without limitation, antimicrobial preservatives and antioxidants. Examples include sorbic acid and its salts, benzoic acid and its salts, calcium propionate, sodium nitrite sodium nitrate, natamycin, sulfites, sulfur dioxide, sodium bisulfite, potassium hydrogen sulfite, disodium EDTA, butylated hydroxyanisole, butylated hydroxytoluene, tert-butylhydroquinone, propyl gallate, ethanol, and methylchloroisothiazolinone.

Food colorings such as FD&C Blue No. 1 (Brilliant Blue FCF), FD&C Blue No. 2 (Indigotine), FD&C Green No. 3 (Fast Green FCF), FD&C Red No. 3 (Erythrosine), FD&C Red No. 40 (Allura Red AC), FD&C Yellow No. 5 (Tartrazine), and FD&C Yellow No. 6 (Sunset Yellow FCF) are contemplated for addition to the formulation to impart a desired color. Coloring, if added, is the last ingredient added before the gelling agent to promote a consistent color throughout the solution.

In an embodiment of the invention, the charged micelles 20 or liposomes 30 that encapsulate the one or more active ingredients 22 are dispersed in a delivery matrix solution. The delivery matrix may be a hydrocolloid or the like.

In one embodiment the hydrocolloid is a hydrogel, which forms an aqueous structure around the micelles 20 or liposomes 30. Many hydrocolloid forming substances are known and can be used in accordance with the invention. Non-limiting examples include cellulose derivatives such as hydroxylethylcellulose, methylcellulose, and hydroxypropyl-methylcellulose; agarose; hyaluronan; pectin; xanthan gum; pullulan; and other polymers either synthetically or naturally derived, with the preferred hydrocolloid forming substance being xanthan gum. The xanthan gum is added to the solution after all other ingredients are added. Since xanthan gum is a gelling agent, adding it prior the addition of any other ingredient will cause the formation of a non-homogenous matrix.

The invention contemplates a method of manufacturing the formulation described herein. The method comprises the steps of: charging water to a heating tank, and heating the water to approximately 78-85° C.; transferring the water to a mixing container; mixing the water at a low speed and adding micelle 20 and/or liposome 30 forming agent and mixing; adding base to the solution; adding active ingredients into the solution; encapsulating active ingredients into micelles 20 and/or liposomes 30; allowing the solution to cool and rest to optimize micelle/liposome 30 formation; adding emulsifying agent to the solution and blending; adding flavor and/or sweetener and/or palatability enhancers and/or coloring to the solution; adding sodium chloride to the solution; adding preservatives to the solution; adding gelling agent to the solution.

The invention also contemplates orally administering the formulation disclosed herein to an individual in need of an energy supplement formulation.

Table 1 summarizes the purposes of ingredients that may be utilized in the current invention.

TABLE 1

| Ingredient | Ingredient Purpose |
|---|---|
| Water, Purified USP | Solvent |
| Xanthan Gum | Gelling agent |
| Genu Pectin 150 USA-SAG TYPE D | Gelling agent |
| Grapeseed Oil | Hydrophobic carrier |
| Phosphatidyl Serine | Forms carrier liposomes |
| Glycerin 99.7% USP | Emulsifying agent |
| Soy Lecithin | Hydrophobic carrier |
| Sodium Hydroxide | Base to create an anionic |
| | surface on the liposome |
| Honey Dew | Flavor oil for |
| | palatability |
| Mixed Berry | Flavor oil for |
| | palatability |
| Mountain Berry | Flavor oil for |
| | palatability |
| Caffeine Anhydrous, USP | Energy |
| Niacin, USP | Part of B-vitamin complex |
| Pyridoxine-Vitamin B6 | Part of B-vitamin complex |
| Folic Acid, USP | Part of B-vitamin complex |
| Vitamin B12-Cyanocobalamin, USP | Part of B-vitamin complex |
| Sucralose USP/NF | Sweetner for palatability |
| Acesulfame Potassium, FCC | Sweetner for palatability |
| Xylitol | Sweetner for palatability |
| Nat & Art Mixed Berry Flavor | Flavor for palatability |
| Sodium Chloride, USP | For palatability |
| Sodium Benzoate | Preservative |
| Potassium Sorbate | Preservative |

### EXAMPLES

The following is an example of an embodiment of the formulation and a method of making the formulation and should not be considered as limiting in any way.

TABLE 2
Batch JB-BED12-D3A

| Ingredient | % Weight |
|---|---|
| Water, Purified USP | 78-82% |
| Genu Pectin 150 USA-SAG TYPE D | 0.5-6% |
| Phosphatidyl Serine | 0.5-3% |
| Glycerin 99.7% USP | 1-3% |
| Sodium Hydroxide | 0.1-0.5% |
| Honey Dew | 0.50-2% |
| Mixed Berry | 0.50-2% |
| Mountain Berry | 0.50-2% |
| Caffeine Anhydrous, USP | 1-5% |
| Niacin, USP | 0.05-0.1% |
| Pyridoxine-Vitamin B6 | 0.5-5% |
| Folic Acid, USP | .005-.05% |
| Vitamin B12-Cyanocobalamin,USP | .005-.05% |
| Sucralose USP/NF | 0.5-1.0% |
| Acesulfame Potassium, FCC | 0.1-0.5% |
| Xylitol | 0.5-1.0% |
| Nat & Art Mixed Berry Flavor | 0.50-2% |
| Sodium Chloride, USP | 0.5-1.0% |
| Sodium Benzoate | .01-.05% |
| Potassium Sorbate | .01-.05% |
| Total: | 100.000% |

An exemplary formulation prepared in accordance with the invention is provided in Table 2. This exemplary formulation comprises the active ingredients Caffeine, Niacin, Pyridoxine, Folic Acid, and Cyanocobalamin, all of which are liposomally encapsulated. Using these active ingredients, the composition functions as an energy supplement formulation delivery solution.

The composition of Table 2 was prepared as follows: purified USP water was heated to approximately 78-85° C. (172-185° F.) in a heating tank. The water was charged to a mixing container, and blended using a high-shear variable speed mixer at 1000 RPM. While blending, Phosphatidyl Serine was charged to the mixing container.

Still blending at the same speed, Sodium Hydroxide was charged to the mixing container. This solution was mixed at 3600 RPM for 2-3 minutes.

The speed of the mixing was returned to 1000 RPM while the following listed ingredients were charged to the mixing container in the following order: 1) Anhydrous Caffeine, 2) Niacin, 3) Pyridoxine, 4) Folic Acid, and 4) Cyanocobalamin. The solution was then blended at 3600 RPM until the appearance of homogeneity was reached.

The mixing was halted, and the solution rested for about 5-7 minutes, allowing the solution to slightly cool. The mixing was resumed at 1000 RPM and the Glycerin was added to the solution. Then the mixing was sped up to 2000 RPM until the appearance of homogeneity was reached.

The solution was then mixed at 1000 RPM, and the following ingredients were added in the following order: 1) Sodium Chloride, 2) Natural & Artificial Mixed berry flavor, 3) Xylitol, 4) Honeydew Flavor oil, 5) Mixed Berry Flavor oil, 6) Mountain Berry flavor oil 7) Sucralose, and 8) Acesulfame Potassium. Once all of these were added to the solution, the speed of mixing was increased to 2000 RPM and blended until the appearance of homogeneity was reached.

Mixing was continued at 1000 RPM and the following ingredients were added to the solution in sequential order: 1) Sodium Benzoate, and 2) Potassium Sorbate. Mixing speed was increased to 2000 RPM and the pectin was added to the solution and blended until the appearance of homogeneity was reached.

The following is another example of an embodiment of the composition and should not be considered as limiting in any way.

TABLE 3

| Ingredient | % Weight |
|---|---|
| Water, Purified USP | 78-82% |
| Xanthan Gum | 0.5-6% |
| Grapeseed Oil | 0.5-3% |
| Glycerin 99.7% USP | 1-3% |
| Soy Lecithin | 1-3% |
| Honey Dew | 0.50-3% |
| Mixed Berry | 0.50-3% |
| Mountain Berry | 0.50-3% |
| Caffeine Anhydrous, USP | 1-5% |
| Niacin, USP | 0.05-0.1% |
| Pyridoxine-Vitamin B6 | 0.5-5% |
| Folic Acid, USP | .005-.05% |
| Vitamin B12-Cyanocobalamin, USP | .005-.05% |
| Sucralose USP/NF | 1-2% |
| Acesulfame Potassium, FCC | 0.1-0.5% |
| Xylitol | 0.5-1.5% |
| Nat & Art Mixed Berry Flavor | 1-2% |
| Sodium Chloride, USP | 0.5-1% |
| Sodium Benzoate | .01-.05% |
| Potassium Sorbate | .01-.05% |
| Total: | 100.000% |

Another exemplary formulation prepared in accordance with the invention is provided in Table 3. This exemplary formulation comprises the active ingredients Anhydrous Caffeine, Niacin, Vitamin B6, Folic Acid, and Vitamin B12, which are contained in a plurality of hydrophobic carriers dispersed in an aqueous medium comprising a hydrocolloid. With these active ingredients, the formulation provides an energy delivery hydrocolloid.

The composition of Table 3 was prepared as follows: purified USP water was heated to approximately 78-85° C. (172-185° F.) in a heating tank. Grapeseed Oil and Glycerin was mixed together for approximately 1-2 minutes to combine. The heated water was charged to a mixing container; and Anhydrous Caffeine, Folic Acid, Niacin, Cyanocobalamin (Vitamin B12), Pyridoxine Vitamin B6), and Acesulfame Potassium were each added into the mixing container with the heated water and blended at high speed for approximately 1-2 minutes.

The mix of Grapeseed Oil and Glycerin were then added into the mixing container with the other ingredients and blended thoroughly at high speed until homogeneous. This mixture is then allowed to sit for 4-6 minutes.

Next, Sodium Chloride, Mixed Berry Flavoring, Mixed Berry Powder, Mountain Berry, Xylitol, Sucralose, and Honeydew Flavoring are added into the mixer and blended thoroughly until homogenous. Sodium Benzoate and Potassium Sorbate are then added and blended until homogenous. Xanthan Gum is finally added and the mixture blended thoroughly until homogenous.

The invention has been described hereinabove with reference to preferred embodiments. Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described. However, the skilled should understand that the methods and materials used and described are examples and may not be the only ones suitable for use in the invention.

Moreover, it should also be understood that any temperature, weight, mass, volume, time interval, pH, salinity, molarity or molality, range, concentration and any other measurements, quantities or numerical figures expressed herein are intended to be approximate and not an exact or critical figure unless expressly stated to the contrary.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the application.

What is claimed is:

1. An oral dosage formulation, comprising:
at least one active ingredient, wherein the at least one active ingredient is contained in a plurality of hydrophobic carriers dispersed in an aqueous medium comprising a hydrocolloid; and
an emulsifier.

2. The formulation of claim 1, wherein the at least one active ingredient is selected from the groups consisting of methylxanthines, caffeine, thiamine, riboflavin, niacin, niacinamide, pantothenic acid, pyridoxine, pyridoxal, pyridoxamine, pyridoxine hydrochloride, biotin, folic acid, cyanocobalamin, guarana, yerba mate, acai, taurine, ginseng, maltodextrin, inositol, carnitine, creatine, glucuronolactone, ginkgo biloba, and combinations thereof.

3. The formulation of claim 1, wherein the at least one active ingredient is about 1% (w/w) to about 15% (w/w) of the formulation.

4. The formulation of claim 1, wherein the at least one active ingredient is selected from the group consisting of caffeine, niacin, pyridoxine, folic acid, and cyanocobalamin, and combinations thereof.

5. The formulation of claim 4, wherein the caffeine is about 1% (w/w) to about 5% (w/w) of the formulation, the niacin is about 0.05% (w/w) to about 0.1% (w/w) of the formulation, the pyridoxine is about 0.5% (w/w) to about 5% (w/w) of the formulation, the folic acid is about 0.005% (w/w) to about 0.05% (w/w) of the formulation, and cyanocobalamin is about 0.005% (w/w) to about 0.05% (w/w) of the formulation.

6. The formulation of claim 1, wherein the hydrophobic carriers are oils.

7. The formulation of claim 1, wherein the hydrophobic carriers are selected from the group consisting of grapeseed oil, lecithin, phosphatidylserine, and combinations thereof.

8. The formulation of claim 1, wherein the hydrophobic carriers are about 0.5% (w/w) to about 3% (w/w) of the formulation.

9. The formulation of claim 1, wherein the emulsifier is selected from the group consisting of sodium stearoyl lactylate, cetearyl alcohol, polysorbates, polyoxyethylene ethers, polyethylene glycol, anisolic compounds, glycerine, and combinations thereof.

10. The formulation of claim 1, wherein the emulsifier is glycerine.

11. The formulation of claim 1, wherein the emulsifier is about 1% (w/w) to about 3% (w/w) of the formulation.

12. The formulation of claim 1, further comprising a vasodilator.

13. The formulation of claim 11, wherein the vasodilator is selected from the group consisting of menthol, menthyl acetate, DDAIP, fatty acid esters, fatty alcohol ethers, ethanol, dimethylsulfoxide, polyethylene glycol monolaurate, sesquiterpenes, terpenoids, sesquiterpenoids, and terpenes.

14. The formulation of claim 11, wherein the vasodilator is menthol.

15. The formulation of claim 11, wherein the vasodilator is about 0.2% (w/w) to about 7% (w/w) of the formulation.

16. The formulation of claim 1, wherein the hydrocolloid is selected from the group consisting of hydroxyethylcellulose, methylcellulose, hydroxypropyl-methylcellulose, chemically modified celluloses, xanthan gum, locust bean gum, carrageenans, gellan gums, polyvinyl alcohol, agarose, hyaluronan, pectin, and pullulan.

17. The formulation of claim 1, wherein the hydrocolloid is xanthan gum.

18. The formulation of claim 1, further comprising a vasostimulant.

19. The formulation of claim 18, wherein the vasostimulant is selected from the group consisting of nicotinic acid, caffeine and combinations thereof.

20. The formulation of claim 18, wherein the vasostimulant is about 0.2% (w/w) to about 7% (w/w) of the formulation.

21. An oral dosage formulation, comprising:
at least one energy stimulating compound, wherein the at least one compound is contained in a plurality of hydrophobic carriers dispersed in an aqueous medium comprising xanthan gum; and
at least one emulsifier.

22. The formulation of claim 21, wherein the at least one energy stimulating compound is selected from the group consisting of caffeine, niacin, pyridoxine, folic acid, and cyanocobalamin, and combinations thereof.
